(19) United States
(12) Patent Application Publication
(10) Pub. No.: US 2026/0158208 A1
(43) Pub. Date: Jun. 11, 2026

(54) DRESSING AND THERAPY SYSTEM

(71) Applicant: Solventum Intellectual Properties Company, Maplewood, MN (US)

(72) Inventors: Andrew J. MCGREGOR, San Antonio, TX (US); Javier GONZALEZ, San Antonio, TX (US); Kamil Kranc, Sutton London (GB); Hannah Lawson, Sutton London (GB)

(21) Appl. No.: 19/150,100

(22) PCT Filed: Jan. 9, 2024
(86) PCT No.: PCT/IB2024/050195

§ 371 Date: Jul. 22, 2025

Related U.S. Application Data
(60) Provisional application No. 63/440,958, filed on Jan. 25, 2023.

(51) Int. Cl. A61M 1/00

(52) U.S. Cl. CPC A61M 1/915; A61M 1/916

(57) ABSTRACT

A dressing for an open abdominal wound is disclosed. The dressing includes a first manifold configured to be disposed within the open abdominal wound. The dressing further includes a traction film attached at least partially to the first manifold and configured to be disposed within the open abdominal wound. The traction film includes a plurality of microstructures extending away from the first manifold and a plurality of through openings. The plurality of microstructures is configured to engage a bottom surface of an abdominal wall around the open abdominal wound. The first manifold is configured to receive a negative pressure from a negative pressure source via the plurality of through openings of the traction film.

### CROSS-REFERENCE TO RELATED APPLICATIONS

This application is a U.S. National Stage Entry of PCT International Application No. PCT/IB2024/050195, filed Jan. 9, 2024, which claims the benefit of priority to U.S. Provisional Application No. 63/440,958, filed on Jan. 25, 2023, which is incorporated herein by reference in its entirety.

### TECHNICAL FIELD

The present disclosure relates generally to a dressing and a therapy system for an open abdominal wound.

### BACKGROUND

Negative pressure wound therapy (NPWT) involves applying a negative pressure to a wound site to promote wound healing. NPWT may be used to treat abdominal wounds due to an abdominal laparotomy. Abdominal laparotomy is a surgical procedure that is performed to gain access to abdominal cavity for a surgery and/or to relieve intra-abdominal pressure by allowing bowels to expand. Such abdominal wounds may require cutting of the fascial layer, which is a thin, fibrous layer of tissue located beneath abdominal muscles that holds abdominal contents (e.g., internal organs and the bowels) together. In some instances, the laparotomy incision is not immediately closed, resulting in an “open abdomen.” Open abdomen may increase a risk of infection and organ disfunction. Therefore, there remains a need for a dressing that expedites closure of the fascial layer (or open abdomen) and promotes healing of abdominal wounds.

### SUMMARY

In a first aspect, the present disclosure provides a dressing for an open abdominal wound. The dressing includes a first manifold. The first manifold is configured to be disposed within the open abdominal wound. The dressing further includes a traction film attached at least partially to the first manifold. The traction film is configured to be disposed within the open abdominal wound. The traction film includes a plurality of microstructures extending away from the first manifold. The traction film further includes a plurality of through openings. The plurality of microstructures is configured to engage a bottom surface of an abdominal wall around the open abdominal wound. The first manifold is configured to receive a negative pressure from a negative pressure source via the plurality of through openings of the traction film.

In a second aspect, the present disclosure provides a dressing for an open abdominal wound. The dressing includes a first manifold. The first manifold is configured to be disposed within the open abdominal wound. The dressing further includes a first adhesive layer disposed on the first manifold. The first adhesive layer includes a plurality of first adhesive through openings. The dressing further includes a second manifold configured to be disposed within the open abdominal wound. The first adhesive layer bonds the second manifold to the first manifold. The dressing further includes a second adhesive layer disposed on the second manifold opposite to the first manifold. The second adhesive layer includes a plurality of second adhesive through openings. The dressing further includes a third manifold disposed on the second manifold opposite to the first manifold. The third manifold is configured to at least partially engage an edge of an abdominal wall around the open abdominal wound. The second adhesive layer bonds the third manifold to the second manifold. The dressing further includes a third adhesive layer disposed on the third manifold opposite to the second manifold. The third adhesive layer includes a plurality of third adhesive through openings. The dressing further includes a drape covering the third manifold. The third adhesive layer bonds the drape to the third manifold. The drape is configured to be attached at least partially to a top surface of the abdominal wall. The drape is further configured to be disposed in fluid communication with a negative pressure source for application of a negative pressure to the third manifold.

In a third aspect, the present disclosure provides a therapy system for an open abdominal wound. The therapy system includes a negative pressure source configured to generate a negative pressure. The therapy system further includes a dressing disposed in fluid communication with the negative pressure source. The dressing includes a first manifold. The first manifold is configured to be disposed within the open abdominal wound. The dressing further includes a traction film attached at least partially to the first manifold. The traction film is configured to be disposed within the open abdominal wound. The traction film includes a plurality of microstructures extending away from the first manifold. The traction film further includes a plurality of through openings. The plurality of microstructures is configured to engage a bottom surface of an abdominal wall around the open abdominal wound. The first manifold receives the negative pressure from the negative pressure source via the plurality of through openings of the traction film.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. In particular, thicknesses of certain layers in proportion to certain other items are exaggerated for ease of illustration and clarity purposes. Like numbers used in the figures refer to like components. However, it will be understood that the use of a number to refer to a component in a given figure is not intended to limit the component in another figure labeled with the same number.

FIG. 1 is a functional block diagram of a therapy system according to an embodiment of the present disclosure;

FIG. 2 is a schematic cross-sectional view of a dressing according to an embodiment of the present disclosure;

FIG. 3 is a schematic cross-sectional view of a traction film of the dressing of FIG. 2 according to an embodiment of the present disclosure;

FIG. 4 is a schematic exploded cross-sectional view of a dressing according to another embodiment of the present disclosure;

FIG. 5 is a schematic plan view of a first manifold of the dressing of FIG. 4 according to an embodiment of the present disclosure;

FIG. 6A is a schematic plan view of a second manifold of the dressing of FIG. 4 according to an embodiment of the present disclosure;

FIG. 6B is a schematic plan view of a third manifold of the dressing of FIG. 4 according to an embodiment of the present disclosure; and

FIG. 7 is a schematic cross-sectional view of a dressing according to another embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” “coupled,” and variations thereof, are used broadly and encompass both direct and indirect attachments, connections, and couplings.

As used herein, the term “moisture vapor transmission rate” or “MVTR” refers to the permissible moisture volume from one side of the substrate web to the other side of the substrate web per area unit (e.g., per square meter) and per time unit (e.g., per one day).

As used herein, the term “configured to” is at least as restrictive as the term “adapted to” and requires actual design intention to perform the specified function rather than mere physical capability of performing such a function.

As used herein, the term “layer” refers to a thickness of a material or blend of materials. Layers may be continuous or discontinuous.

As used herein, the term “adhesive layer” refers to a layer of adhesive material disposed on one or more layers to promote an adhesion of the one or more layers to each other or to another surface. Adhesive layers may be patterned.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

As used herein, the term “open abdominal wound” refers to an abdominal incision that includes an incision in the fascial layer to access the abdominal cavity. The fascial layer is a layer of tissue that surrounds and supports the abdominal contents (e.g., the bowels and the internal organs). The phrase “open abdomen” refers to conditions in which a deep abdominal wound is left open (e.g., the abdominal incision is not resealed) for a period of time. For example, the abdomen may be left open to accommodate swelling of the bowels and/or other abdominal contents (e.g., internal organs). The abdomen may also be left open in conditions in which further surgery in the abdominal cavity is required.

As used herein, the term “through openings” and variations thereof when used in conjunction with a layer or a film refers to holes, vents, slits, slots, perforations, notches, punctures, orifices, openings, inlets, and channels, extending fully through the layer or the film.

The present disclosure relates to a dressing for an open abdominal wound. The dressing includes a first manifold. The first manifold is configured to be disposed within the open abdominal wound. The dressing further includes a traction film attached at least partially to the first manifold. The traction film is configured to be disposed within the open abdominal wound. The traction film includes a plurality of microstructures extending away from the first manifold. The traction film further includes a plurality of through openings. The plurality of microstructures is configured to engage a bottom surface of an abdominal wall around the open abdominal wound. The first manifold is configured to receive a negative pressure from a negative pressure source via the plurality of through openings of the traction film.

The dressing of the present disclosure may expedite closure of the primary fascia and the open abdominal wound. Specifically, upon application of the negative pressure, the first manifold may collapse in a collapse plane that is substantially perpendicular to a thickness of the dressing. This collapse may generate a medial force that is applied to the traction film in the collapse plane. The plurality of microstructures may engage with the bottom surface of the abdominal wall and at least partially transfer the medial force to the abdominal wall. Thus, the medial force may be applied to the abdominal wall by the dressing. As a result, the dressing may facilitate and expedite healing of the open abdominal wound. Faster primary fascial closure may improve overall patient outcomes and may significantly lower patient mortality.

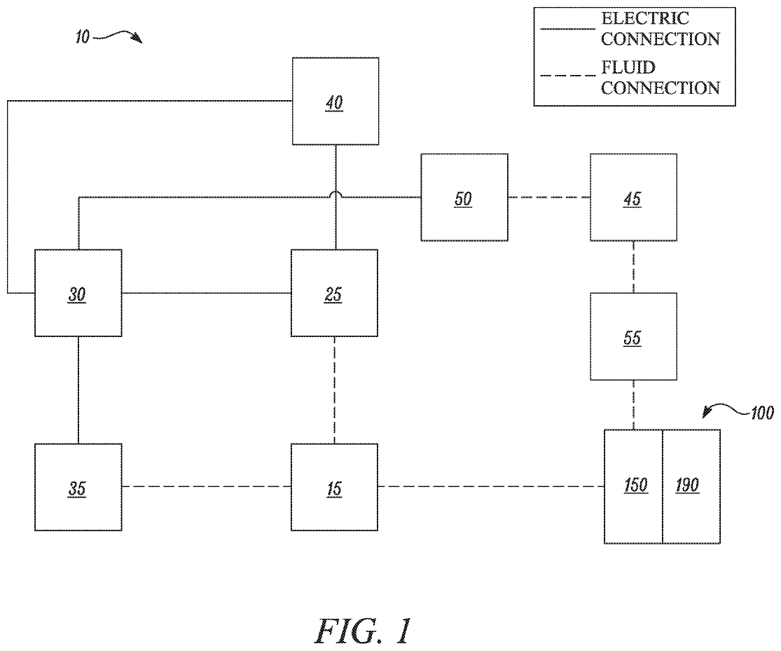

Referring now to the Figures, FIG. 1 illustrates a schematic functional block diagram of a therapy system 10 for an open abdominal wound according to an embodiment of the present disclosure.

The therapy system 10 includes a negative pressure source 25. The therapy system 10 may further include one or more distribution components. A distribution component may be detachable and may be disposable, reusable, and/or recyclable. A dressing, such as a dressing 100, and a fluid container, such as a container 15, are examples of distribution components that may be associated with the therapy system 10. Specifically, the therapy system 10 includes the dressing 100. As shown in FIG. 1, the dressing 100 may include a tissue interface 190, a cover or drape 150, or both in some embodiments.

A fluid conductor is another example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include interfaces or fluid ports to facilitate coupling and de-coupling other components. In some embodiments, for example, a dressing interface may facilitate coupling a fluid conductor to the dressing 100. For example, such a dressing interface may be a SENSAT.R.A.C.™ Pad available from Kinetic Concepts, Inc. of San Antonio, Tex.

The therapy system 10 may further include a regulator or controller, such as a controller 30. Additionally, the therapy system 10 may include sensors to measure operating parameters and provide feedback signals to the controller 30 indicative of the operating parameters. As shown in FIG. 1, for example, the therapy system 10 may include a first sensor 35 and a second sensor 40, and each of the first sensor 35 and the second sensor 40 may be communicably coupled to the controller 30.

In some embodiments, the therapy system 10 may further include a source of instillation solution. For example, a solution source 45 may be fluidly coupled to the dressing 100, as shown in FIG. 1. The solution source 45 may be fluidly coupled to a positive pressure source, such as a positive-pressure source 50, a negative pressure source, such as the negative pressure source 25, or both in some embodiments. A regulator, such as an instillation regulator 55, may also be fluidly coupled to the solution source 45 and the dressing 100 to ensure proper dosage of instillation solution (e.g., saline) to a tissue site. For example, the instillation regulator 55 may comprise a piston that can be pneumatically actuated by the negative pressure source 25 to draw instillation solution from the solution source during a negative pressure interval and to instill the solution to a dressing during a venting interval. Additionally or alternatively, the controller 30 may be coupled to the negative pressure source 25, the positive-pressure source 50, or both, to control dosage of instillation solution to a tissue site. In some embodiments, the instillation regulator 55 may also be fluidly coupled to the negative pressure source 25 through the dressing 100, as shown in FIG. 1.

Some components of the therapy system 10 may be housed within or used in conjunction with other components, such as sensors, processing units, alarm indicators, memory, databases, software, display devices, or user interfaces that further facilitate therapy. For example, in some embodiments, the negative pressure source 25 may be combined with the controller 30, the solution source 45, and other components into a therapy unit.

In general, components of the therapy system 10 may be coupled directly or indirectly. For example, the negative pressure source 25 may be directly coupled to the container 15 and may be indirectly coupled to the dressing 100 through the container 15. Coupling may include fluid, mechanical, thermal, electrical (wired or wireless), or chemical coupling (such as a chemical bond), or some combination of coupling in some contexts. For example, the negative pressure source 25 may be electrically coupled to the controller 30 and may be fluidly coupled to one or more distribution components to provide a fluid path to a tissue site. In some embodiments, components may also be coupled by virtue of physical proximity, being integral to a single structure, or being formed from the same piece of material.

A negative pressure supply, such as the negative pressure source 25, may be a reservoir of air at a negative pressure or may be a manual or electrically powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative pressure source 25 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 15 is representative of a container, canister, pouch, absorbent, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative pressure therapy.

A controller, such as the controller 30, may be a microprocessor or computer programmed to operate one or more components of the therapy system 10, such as the negative pressure source 25. In some embodiments, for example, the controller 30 may be a microcontroller, which generally comprises an integrated circuit containing a processor core and a memory programmed to directly or indirectly control one or more operating parameters of the therapy system 10. Operating parameters may include the power applied to the negative pressure source 25, the pressure generated by the negative pressure source 25, or the pressure distributed to the tissue interface 190, for example. The controller 30 is also preferably configured to receive one or more input signals, such as a feedback signal, and programmed to modify one or more operating parameters based on the input signals.

Sensors, such as the first sensor 35 and the second sensor 40, are generally known in the art as any apparatus operable to detect or measure a physical phenomenon or property, and generally provide a signal indicative of the phenomenon or property that is detected or measured. For example, the first sensor 35 and the second sensor 40 may be configured to measure one or more operating parameters of the therapy system 10. In some embodiments, the first sensor 35 may be a transducer configured to measure pressure in a pneumatic pathway and convert the measurement to a signal indicative of the pressure measured. In some embodiments, for example, the first sensor 35 may be a piezo-resistive strain gauge. The second sensor 40 may optionally measure operating parameters of the negative pressure source 25, such as a voltage or current, in some embodiments. Preferably, the signals from the first sensor 35 and the second sensor 40 are suitable as an input signal to the controller 30, but some signal conditioning may be appropriate in some embodiments. For example, the signal may need to be filtered or amplified before it can be processed by the controller 30. Typically, the signal is an electrical signal, but may be represented in other forms, such as an optical signal.

As noted above, the dressing 100 may include the tissue interface 190, the drape 150, or both in some embodiments. The tissue interface 190 can be generally adapted to partially or fully contact a tissue site. The tissue interface 190 may take many forms, and may have many sizes, shapes, or thicknesses, depending on a variety of factors, such as the type of treatment being implemented or the nature and size of a tissue site. For example, the size and shape of the tissue interface 190 may be adapted to the contours of deep and irregular shaped tissue sites. Any or all of the surfaces of the tissue interface 190 may have an uneven, coarse, or jagged profile.

The tissue interface 190 may include one or more manifolds. A manifold in this context may include a means for collecting or distributing fluid across the tissue interface 190 under pressure. For example, a manifold may be adapted to receive negative pressure from a source and distribute negative pressure through multiple apertures across the tissue interface 190, which may have the effect of collecting fluid from across a tissue site and drawing the fluid toward the source. In some embodiments, the fluid path may be reversed, or a secondary fluid path may be provided to facilitate delivering fluid, such as fluid from a source of instillation solution, across a tissue site.

A manifold may include a plurality of pathways, which can be interconnected to improve distribution or collection of fluids. In some embodiments, a manifold may include a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively include projections that form interconnected fluid pathways. For example, a manifold may be molded to provide surface projections that define interconnected fluid pathways.

In some embodiments, a manifold may comprise or consist essentially of reticulated foam having pore sizes and free volume that may vary according to needs of a prescribed therapy. For example, reticulated foam having a free volume of at least 40%, at least 50%, at least 60%, at least 70%, at least 80%, or at least 90%, may be suitable for many therapy applications, and foam having an average pore size in a range of 400-600 microns (40-50 pores per inch) may be particularly suitable for some types of therapy. The tensile strength of the tissue interface 190 may also vary according to needs of a prescribed therapy. For example, the tensile strength of foam may be increased for instillation of topical treatment solutions. The 25% compression load deflection of the tissue interface 190 may be at least 0.35 pounds per square inch, and the 65% compression load deflection may be at least 0.43 pounds per square inch. In some embodiments, the tensile strength of a manifold may be at least 10 pounds per square inch. A manifold may have a tear strength of at least 2.5 pounds per inch. In some embodiments, a manifold may be foam comprised of polyols such as polyester or polyether, isocyanate such as toluene diisocyanate, and polymerization modifiers such as amines and tin compounds. In some examples, a manifold may be reticulated polyurethane foam such as found in GRANUFOAM™ dressing or V.A.C. VERAFLO™ dressing, both available from Kinetic Concepts, Inc. of San Antonio, Tex.

Other suitable materials for the one or more manifold may include non-woven fabrics (Libeltex, Freudenberg), three-dimensional (3D) polymeric structures (molded polymers, embossed and formed films, and fusion bonded films [Supracore]), and mesh, for example.

In some examples, a manifold may include a 3D textile, such as various textiles commercially available from Baltex, Muller, and Heathcoates. A 3D textile of polyester fibers may be particularly advantageous for some embodiments. For example, a manifold may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 mm may be particularly advantageous for some embodiments. Such a puncture-resistant fabric may have a warp tensile strength of about 330-340 kilograms and a weft tensile strength of about 270-280 kilograms in some embodiments. Another particularly suitable material may be a polyester spacer fabric having a weight of about 470 grams per square meter, which may have a thickness of about 4-5 mm in some embodiments. Such a spacer fabric may have a compression strength of about 20-25 kilopascals (at 40% compression). Additionally or alternatively, a manifold may include a material having substantial linear stretch properties, such as a polyester spacer fabric having 2-way stretch and a weight of about 380 grams per square meter. A suitable spacer fabric may have a thickness of about 3-4 mm, and may have a warp and weft tensile strength of about 30-40 kilograms in some embodiments. The fabric may have a close-woven layer of polyester on one or more opposing faces in some examples. In some embodiments, a woven layer may be advantageously disposed on a manifold to face a tissue site.

The thickness of a manifold may also vary according to needs of a prescribed therapy. For example, the thickness of a manifold may be decreased to reduce tension on peripheral tissue. The thickness of a manifold can also affect the conformability of the tissue interface 190. In some embodiments, a manifold thickness, e.g., for a suitable foam, may be in a range of about 3 mm to 10 mm, preferably about 6 mm to about 8 mm. Fabrics, including suitable 3D textiles and spacer fabrics, may have a thickness in a range of about 2 mm to about 8 mm.

A manifold disclosed herein may be either hydrophobic or hydrophilic. In an example in which a manifold may be hydrophilic, the manifold may also wick fluid away from a tissue site, while continuing to distribute negative pressure to the tissue site. The wicking properties of a manifold may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, a manifold may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. A manifold may further serve as a scaffold for new cell-growth, or a scaffold material may be used in conjunction with a manifold to promote cell-growth. A scaffold is generally a substance or structure used to enhance or promote the growth of cells or formation of tissue, such as a three-dimensional porous structure that provides a template for cell growth. Some examples of scaffold materials include calcium phosphate, collagen, PLA/PGA, coral hydroxy apatites, carbonates, or processed allograft materials. Additional embodiments of manifolds for use in the dressing 100 are discussed further herein.

In addition to the tissue interface 190, the dressing 100 may further include the drape 150. In some embodiments, the drape 150 may provide a bacterial barrier and protection from physical trauma. The drape 150 may also be constructed from a material that can reduce evaporative losses and provide a fluid seal between two components or two environments, such as between a therapeutic environment and a local external environment. The drape 150 may include, for example, an elastomeric film or membrane that can provide a seal adequate to maintain a negative pressure at a tissue site for a given negative pressure source. The drape 150 may have a high moisture-vapor transmission rate (MVTR) in some applications. For example, the MVTR may be at least 250 grams per square meter per twenty-four hours in some embodiments, measured using an upright cup technique according to ASTM E 96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the drape 150 may be a non-porous polymer drape or film, such as a polyurethane film, that is permeable to water vapor but impermeable to liquid. Such drapes typically have a thickness in the range of 25-50 microns. For permeable materials, the permeability generally should be low enough that a desired negative pressure may be maintained. The drape 150 may include, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn. ; polyurethane (PU) drape, commercially available from Avery Dennison Corporation, Pasadena, Calif. ; polyether block polyamide copolymer (PEBAX), for example, from Arkema S. A., Colombes, France; and Inspire 2301 and Inpsire 2327 polyurethane films, commercially available from Coveris Advanced Coatings, Wrexham, United Kingdom. In some embodiments, the drape 150 may comprise INSPIRE 2301 having an MVTR (upright cup technique) of 2600 g/m2/24 hours and a thickness of about 30 microns.

An attachment device may be used to attach the drape 150 to an attachment surface, such as undamaged epidermis, a gasket, or another cover. The attachment device may take many forms. For example, an attachment device may be a medically acceptable, pressure-sensitive adhesive configured to bond the drape 150 to epidermis around a tissue site. In some embodiments, for example, some or all of the drape 150 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 45 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The dressings disclosed herein may be used with negative pressure therapy. In some embodiments, the dressing 100 disclosed herein may be used for at least 5, 6, 7, 8, 9, 10, 11 or 12 days to promote granulation and/or minimize tissue in-growth with a source of negative pressure. For example, the dressing 100 disclosed herein may remain on a tissue site, such as a surface wound, for at least 5 to 7 days.

In operation, the tissue interface 190 may be placed within, over, on, or otherwise proximate to a tissue site. If the tissue site is a wound, for example, the tissue interface 190 may partially or completely fill the wound, or it may be placed over the wound. The drape 150 may be placed over the tissue interface 190 and sealed to an attachment surface near a tissue site. For example, the drape 150 may be sealed to undamaged epidermis peripheral to a tissue site. Thus, the dressing 100 can provide a sealed therapeutic environment proximate to a tissue site, substantially isolated from the external environment, and the negative pressure source 25 can reduce pressure in the sealed therapeutic environment.

The fluid mechanics of using a negative pressure source to reduce pressure in another component or location, such as within a sealed therapeutic environment, can be mathematically complex. However, the basic principles of fluid mechanics applicable to negative pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative pressure source, and this descriptive convention should not be construed as a limiting convention.

Negative pressure applied across the tissue site through the tissue interface 190 in the sealed therapeutic environment can induce macro-strain and micro-strain in the tissue site. Negative pressure can also remove exudate and other fluid from a tissue site, which can be collected in container 15.

In some embodiments, the controller 30 may receive and process data from one or more sensors, such as the first sensor 35. The controller 30 may also control the operation of one or more components of the therapy system 10 to manage the pressure delivered to the tissue interface 190. In some embodiments, controller 30 may include an input for receiving a desired target pressure and may be programmed for processing data relating to the setting and inputting of the target pressure to be applied to the tissue interface 190. In some example embodiments, the target pressure may be a fixed pressure value set by an operator as the target negative pressure desired for therapy at a tissue site and then provided as input to the controller 30. The target pressure may vary from tissue site to tissue site based on the type of tissue forming a tissue site, the type of injury or wound (if any), the medical condition of the patient, and the preference of the attending physician. After selecting a desired target pressure, the controller 30 can operate the negative pressure source 25 in one or more control modes based on the target pressure and may receive feedback from one or more sensors to maintain the target pressure at the tissue interface 190.

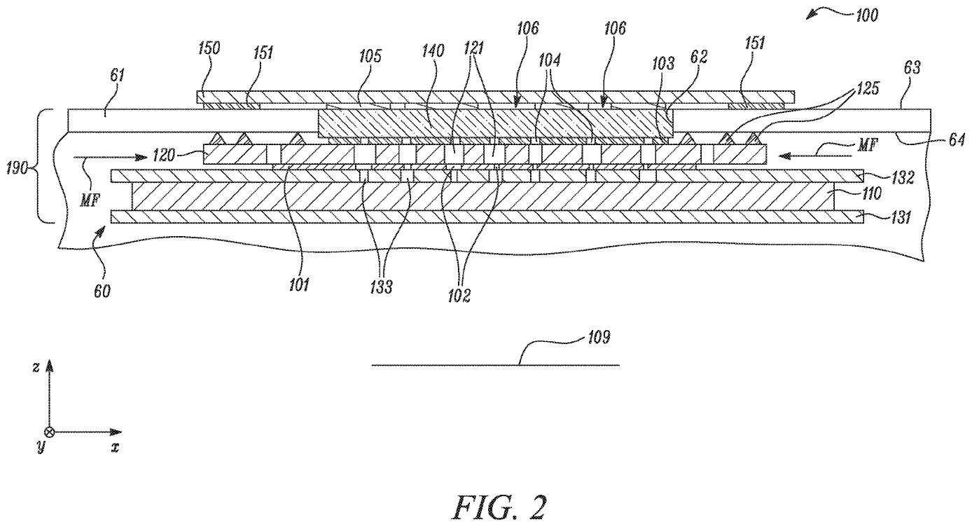

FIG. 2 illustrates a schematic cross-sectional view of the dressing 100 for an open abdominal wound 60 according to an embodiment of the present disclosure.

The dressing 100 defines mutually orthogonal x, y, and z-axes. The x-axis is defined along a length of the dressing 100, while the y-axis is defined along a breadth of the dressing 100. The z-axis is defined along a thickness of the dressing 100.

As shown in FIG. 2, the open abdominal wound 60 may define an opening in an abdominal wall 61. The abdominal wall 61 may include a top surface 63, a bottom surface 64 opposite to the top surface 63, and an edge 62 defining the opening. The dressing 100 may be at least partially disposed within the open abdominal wound 60. More specifically, the tissue interface 190 of the dressing may be at least partially disposed within the open abdominal wound 60. The abdominal wall 61 is shown in FIG. 2 for illustrative purposes only. It may be noted that the abdominal wall 61 is not a part of the dressing 100. The tissue interface 190 of the dressing 100 does not include the abdominal wall 61.

The dressing 100 includes a first manifold 110. The first manifold 110 is configured to be disposed within the open abdominal wound 60. The first manifold 110 may be disposed adjacent to the internal organs and the bowels of a patient.

The dressing 100 further includes a traction film 120. The traction film 120 is configured to be disposed within the open abdominal wound 60. The traction film 120 is attached at least partially to the first manifold 110. The traction film 120 may be attached at least partially to the first manifold 110 by any suitable method and technique, such as adhesive lamination, heat lamination, etc. As shown in FIG. 2A, the dressing 100 may further include a first adhesive layer 101 at least partially attaching the traction film 120 to the first manifold 110. The first adhesive layer 101 may be permeable. The first adhesive layer 101 may include a plurality of first adhesive through openings 102. In some embodiments, the first adhesive layer 101 may be patterned.

The traction film 120 includes a plurality of microstructures 125 extending away from the first manifold 110. As shown in FIG. 2, the plurality of microstructures 125 may extend toward the abdominal wall 61. The plurality of microstructures 125 is configured to engage the bottom surface 64 of the abdominal wall 61 around the open abdominal wound 60. Upon placement of the dressing 100 at least partially within the open abdominal wound 60, the plurality of microstructures 125 may frictionally engage with the bottom surface 64 of the abdominal wall 61.

The traction film 120 further includes a plurality of through openings 121. The plurality of through openings 121 may be, for example, holes, vents, slits, slots, perforations, notches, punctures, orifices, openings, inlets, channels, extending fully through the traction film 120.

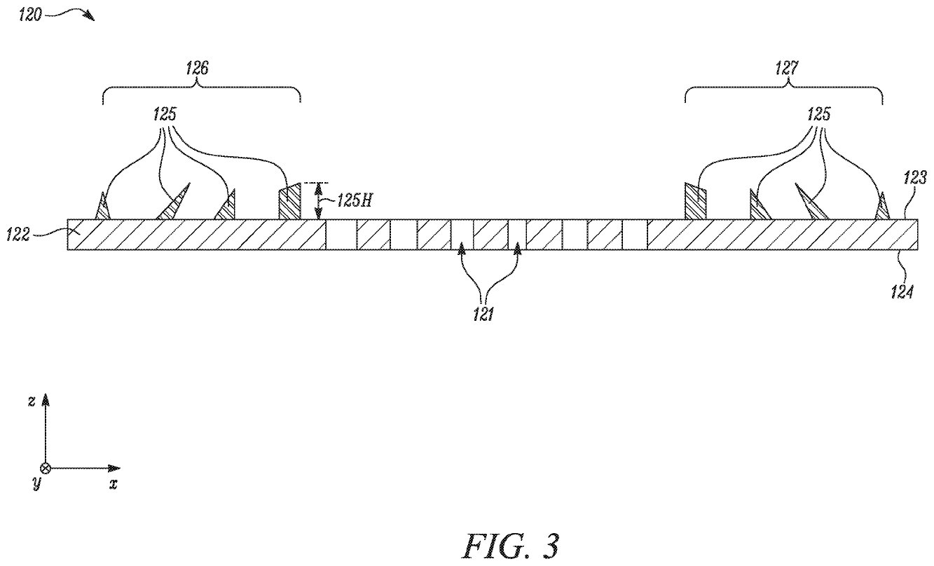

FIG. 3 illustrates a schematic cross-sectional view of the traction film 120 according to an embodiment of the present disclosure.

Referring to FIGS. 2 and 3, the traction film 120 may further include a backing layer 122. The backing layer 122 may include a first major surface 123 and a second major surface 124 opposite to the first major surface 123. The backing layer 122 may include the plurality of through openings 121 of the traction film 120. The plurality of through openings 121 may extend from the first major surface 123 to the second major surface 124 of the backing layer 122. The backing layer 122 may include a polymer, such as polyethylene, polypropylene, and so forth.

The second major surface 124 of the backing layer 122 may face the first manifold 110. Further, each of the plurality of microstructures 125 may extend from the first major surface 123. The plurality of microstructures 125 may extend from the first major surface 123 opposite to the second major surface 124.

The plurality of microstructures 125 may be made from any suitable material using any suitable method. The plurality of microstructures 125 may be made from, for example, polymers (e.g., UV-cured acrylates) and ceramics (e.g., alumina or zirconia).

In some embodiments, the plurality of microstructures 125 may be manufactured separately from the backing layer 122 and subsequently attached to the backing layer 122. In some embodiments, the plurality of microstructures 125 may be formed on the first major surface 123 of the backing layer 122 as discrete structures (e.g., conical microstructures) or continuous structures (e.g., prismatic ridges). In some embodiments, the plurality of microstructures 125 may be integral with the backing layer 122. In some embodiments, the plurality of microstructures 125 may be micropatterned on the backing layer 122.

The plurality of microstructures 125 may include any shape suitable for engaging the bottom surface 64 of the abdominal wall 61. In some embodiments, at least some microstructures 125 from the plurality of microstructures 125 may include a tetrahedron shape, a pyramid shape, a conical shape, or a prismatic shape.

Each of the plurality of microstructures 125 may include a height 125H. The height 125H may extend along the z-axis. The height 125H may be defined between the first major surface 123 of the backing layer 122 and a topmost edge of the microstructure 125. In some embodiments, the height 125H may range from 1 micron to 1000 microns. In some embodiments, the height 125H may range from 10 micron to 500 microns.

The plurality of microstructures 125 may further include a plurality of sets of microstructures radially spaced apart from each other. Specifically, as shown in FIG. 3, the plurality of microstructures 125 may include a first set of microstructures 126 and a second set of microstructures 127. The second set of microstructures 127 may be radially spaced apart from the first set of microstructures 126.

Referring to FIG. 2, the dressing 100 may further include a first visceral protective layer (VPL) film 131 disposed on the first manifold 110 opposite to the traction film 120. The first VPL film 131 may be configured to engage, cover, and protect the abdominal contents.

The dressing 100 may further include a second VPL film 132 disposed on the first manifold 110 opposite to the first VPL film 131, such that the first VPL film 131 and the second VPL film 132 together encapsulate the first manifold 110. The first VPL film 131 and the second VPL film 132 may be attached to the first manifold 110, for example, via a permeable adhesive layer (not shown). In some embodiments, the second VPL film 132 may include a plurality of film through openings 133. While not illustrated in FIG. 2, in some embodiments, the first VPL film 131 may also include a plurality of film through openings.

The first VPL film 131 and/or the second VPL film 132 may be made of a material that is fluid-impermeable and intended to not irritate a patient's fascia and internal organs. For example, in some embodiments, the first VPL film 131 and/or the second VPL film 132 may include polyurethane.

The dressing 100 may further optionally include a second manifold 140. The second manifold 140 may be disposed adjacent to the traction film 120. In the illustrated embodiment of FIG. 2, the second manifold 140 is disposed on the traction film 120 opposite to the first manifold 110. The second manifold 140 may be at least partially attached to the traction film 120. The second manifold 140 may be attached at least partially to the traction film 120 by any suitable method and technique, such as adhesive lamination, heat lamination, etc. As shown in FIG. 2, the dressing 100 may further include a second adhesive layer 103 at least partially attaching the second manifold 140 to the traction film 120. The second adhesive layer 103 may be permeable. The second adhesive layer 103 may include a plurality of second adhesive through openings 104. In some embodiments, the second adhesive layer 103 may be patterned. In the illustrated embodiment of FIG. 2, the second manifold 140 may be configured to at least partially engage the edge 62 of the abdominal wall.

In the illustrated embodiment of FIG. 2, the tissue interface 190 of the dressing 100 includes the first manifold 110, the second manifold 140, the traction film 120, the first VPL film 131, and the second VPL film 132.

As noted above, the dressing 100 may further include the drape 150. The drape 150 may cover the traction film 120. The drape 150 may be attached at least partially to the traction film 120. The drape 150 may be directly or indirectly attached at least partially to the traction film 120. For example, in some embodiments, the second manifold 140 may be omitted from the dressing 100 and the second adhesive layer 103 may directly attach the drape 150 to the traction film 120. However, in the illustrated embodiment of FIG. 2, the dressing 100 further includes a third adhesive layer 105 at least partially attaching the drape 150 to the second manifold 140, and the drape 150 is indirectly attached at least partially to the traction film 120 via the second manifold 140. The third adhesive layer 105 may be permeable. The third adhesive layer 105 may include a plurality of third adhesive through openings 106. In some embodiments, the third adhesive layer 105 may be patterned.

The drape 150 may be configured to be attached at least partially to the top surface 63 of the abdominal wall 61. As shown in FIG. 1, the dressing 100 may further include a skin adhesive 151 disposed adjacent to an edge of the drape 150. The skin adhesive 151 may be configured to at least partially attach the drape 150 to the top surface 63 of the abdominal wall 61. The skin adhesive 151 may include, for example, a hydrocolloid adhesive. Alternatively or in combination, the skin adhesive 151 may include acrylic formulations.

The drape 150 may be further configured to be disposed in fluid communication with the negative pressure source 25 (shown in FIG. 1) for application of the negative pressure to the first manifold 110. For example, the dressing 100 may include a pressure interface (not shown) configured to fluidly couple the drape 150 with the negative pressure source 25. The drape 150 may include one or more ports that interface with the pressure interface.

The first manifold 110 is configured to receive the negative pressure from the negative pressure source 25 (shown in FIG. 1) via the plurality of through openings 121 of the traction film 120. Specifically, the first manifold 110 receives the negative pressure from the negative pressure source 25 via the plurality of through openings 121 of the traction film 120.

Upon application of the negative pressure, the first manifold 110 and/or the second manifold 140 may collapse in a collapse plane 109, thereby generating a medal force MF along the collapse plane 109. In embodiments where the second manifold 140 is omitted, the first manifold 110 may generate the medal force MF along the collapse plane 109. In some embodiments, the first manifold 110 and the second manifold 140 may both collapse in the collapse plane 109, and together generate the medal force MF along the collapse plane 109.

Specifically, upon application of the negative pressure to the first manifold 110, the first manifold 110 and/or the second manifold 140 may collapse in a collapse plane 109 that is substantially perpendicular to a thickness of the dressing 100, such that the medial force MF is applied to the traction film 120 along the collapse plane 109. The first adhesive layer 101 and/or the second adhesive layer 103 may facilitate application of the medial force MF to the traction film 120.

The thickness of the dressing 100 is defined along the z-axis. The collapse plane 109 may be aligned with the x-y plane. Further, the medial force MF is depicted by opposing arrows in FIG. 2. The plurality of microstructures 125 may be configured to at least partially transfer the medial force MF to the abdominal wall 61. Specifically, as the plurality of microstructures 125 may engage with the bottom surface 64 of the abdominal wall 61, the plurality of microstructures 125 may at least partially transfer the medial force MF to the abdominal wall 61.

Moreover, collapse of the first manifold 110 and/or the second manifold 140 in the collapse plane 109 may further apply the medial force MF to the drape 150 along the collapse plane 109. The third adhesive layer 105 may facilitate application of the medial force MF to the drape 150.

The drape 150 may be configured to at least partially transfer the medial force MF to the abdominal wall 61. Specifically, as the drape 150 may be attached to the top surface 63 of the abdominal wall 61, the drape 150 may at least partially transfer the medial force MF to the abdominal wall 61. Therefore, in some embodiments, the traction film 120 and the drape 150 may together at least partially transfer the medial force MF to the abdominal wall 61.

The dressing 100 may apply the medial force MF to the abdominal wall 61. The medial force MF applied to the abdominal wall 61 by the dressing 100 may expedite closure of the primary fascia and the open abdominal wound 60. As a result, the dressing 100 may facilitate and expedite healing of the open abdominal wound 60.

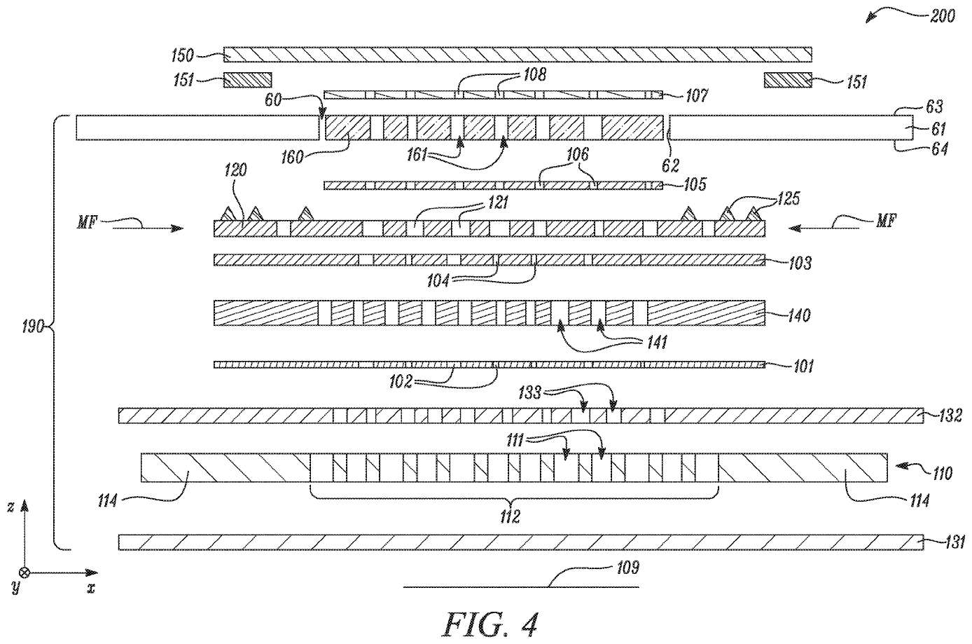

FIG. 4 illustrates a schematic exploded cross-sectional view of a dressing 200 for the open abdominal wound 60 according to another embodiment of the present disclosure. The dressing 200 is similar to the dressing 100 of FIG. 2, with like elements designated by like reference characters. However, the dressing 200 includes a different configuration of the tissue interface 190 as compared to the dressing 100. Further, the abdominal wall 61 is shown in FIG. 4 for illustrative purposes only. It may be noted that the abdominal wall 61 is not a part of the dressing 200. The tissue interface 190 of the dressing 200 does not include the abdominal wall 61.

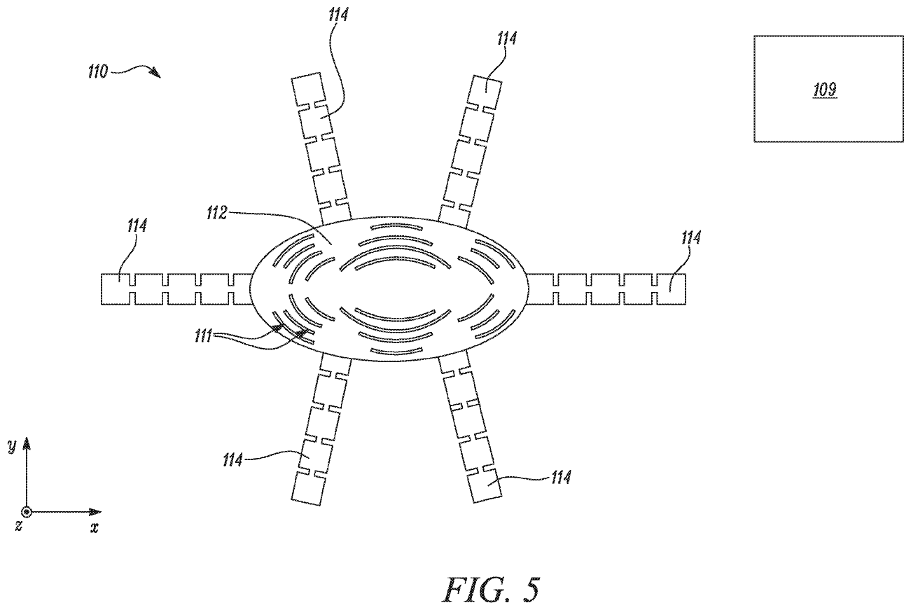

Specifically, the first manifold 110 may include a plurality of first through openings 111. The plurality of first through openings 111 may facilitate collapse of the first manifold 110 in the collapse plane 109 of the dressing 200 upon application of the negative pressure to the first manifold 110. The first manifold 110 is also shown in FIG. 5.

Referring to FIGS. 4 and 5, the first manifold 110 may further include a central portion 112. In some embodiments, the central portion 112 may include the plurality of first through openings 111. While the central portion 112 is shown to have a generally oval shape in FIG. 5, it may be noted that the central portion 112 may have any suitable shape, such as circular, triangular, polygonal, and the like, based on desired application attributes. Further, the plurality of first through openings 111 may be generally elongate and curved. The plurality of first through openings 111 may also have other suitable shapes, such as circular, oblong, triangular, polygonal, etc.

The first manifold 110 may further include a plurality of conduits 114 spaced apart from each other and extending outwardly from the central portion 112. In the illustrated embodiment of FIG. 5, the first manifold 110 includes six conduits 114 extending from the central portion 112. The plurality of conduits 114 may facilitate dispensing of instillation fluid (e.g., from the solution source 45 shown in FIG. 1) to the abdominal cavity so as to prevent drying out of the abdominal contents.

As shown in FIG. 4, in some embodiments, the plurality of film through openings 133 of the second VPL film 132 may be in a one-to-one correspondence with the plurality of first through openings 111 of the first manifold 110.

In the illustrated embodiment of FIG. 4, the second manifold 140 is disposed between the first manifold 110 and the traction film 120. Further, the second manifold 140 is configured to be disposed within the open abdominal wound 60. The second manifold 140 may further include a plurality of second through openings 141. The plurality of second through openings 141 may facilitate collapse of the second manifold 140 in the collapse plane 109 of the dressing 200 upon application of the negative pressure to the second manifold 140.

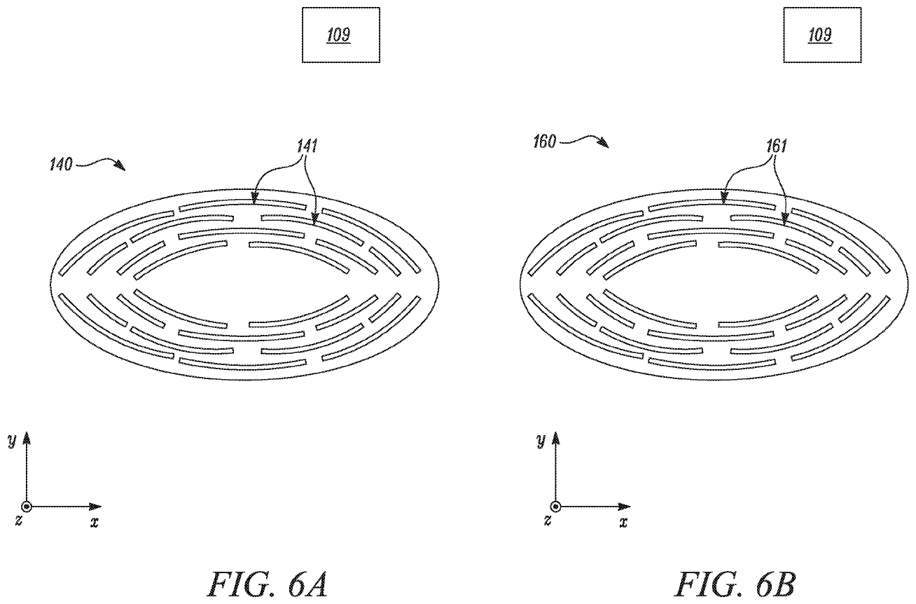

The second manifold 140 is also illustrated in FIG. 6A. While the second manifold 140 is shown to have a generally oval shape in FIG. 6A, it may be noted that the second manifold 140 may have any suitable shape, such as circular, triangular, polygonal, and the like, based on desired application attributes. Further, the plurality of second through openings 141 may be generally elongate and curved. The plurality of second through openings 141 may also have other suitable shapes, such as circular, oblong, triangular, polygonal, etc.

In the illustrated embodiment of FIG. 4, the dressing 200 further includes a third manifold 160 disposed on the traction film 120 opposite to the second manifold 140. Further, the third manifold 160 is configured to at partially engage the edge 62 of the abdominal wall 61. The third manifold 160 may further include a plurality of third through openings 161. The plurality of third through openings 161 may facilitate collapse of the third manifold 160 in the collapse plane 109 of the dressing 200 upon application of the negative pressure to the third manifold 160. In some embodiments, the plurality of microstructures 125 may at least partially surround the third manifold 160.

The third manifold 160 is also illustrated in FIG. 6B. While the third manifold 160 is shown to have a generally oval shape in FIG. 6B, it may be noted that the third manifold 160 may have any suitable shape, such as circular, triangular, polygonal, and the like, based on desired application attributes. Further, the plurality of third through openings 161 may be generally elongate and curved. However, the plurality of third through openings 161 may also have other suitable shapes, such as circular, oblong, triangular, polygonal, etc. In some embodiments, each of the first manifold 110, the second manifold 140, and the third manifold 160 may include a foam.

In the illustrated embodiment of FIG. 4, the tissue interface 190 of the dressing 200 includes the first manifold 110, the second manifold 140, the third manifold 160, the traction film 120, the first VPL film 131, and the second VPL film 132.

Referring to FIG. 4, the first adhesive layer 101 may be at least partially disposed between the first manifold 110 and the second manifold 140. The first adhesive layer 101 may at least partially attach the second manifold 140 to the first manifold 110. Further, the second adhesive layer 103 may be at least partially disposed between the second manifold 140 and the traction film 120. The second adhesive layer 103 may at least partially attach the traction film 120 to the second manifold 140. Moreover, the third adhesive layer 105 may be disposed between the traction film 120 and the third manifold 160. The third adhesive layer 105 may at least partially attach the third manifold 160 to the traction film 120. The dressing 200 may further include a fourth adhesive layer 107 disposed between the third manifold 160 and the drape 150. The fourth adhesive layer 107 may be permeable. The fourth adhesive layer 107 may include a plurality of fourth adhesive through openings 108. In some embodiments, the fourth adhesive layer 107 may be patterned. The fourth adhesive layer 107 may at least partially attach the drape 150 to the third manifold 160.

Upon application of the negative pressure, each of the first manifold 110, the second manifold 140, and the third manifold 160 may collapse in the collapse plane 109 that is substantially perpendicular to the thickness of the dressing 200, such that the medial force MF is applied to the traction film 120 along the collapse plane 109. The first adhesive layer 101, the second adhesive layer 103, and the third adhesive layer 105 may facilitate application of the medial force MF to the traction film 120.

Further, upon collapse of each of the first manifold 110, the second manifold 140, and the third manifold 160 in the collapse plane 109, the medial force MF may be further applied to the drape 150 along the collapse plane 109. The fourth adhesive layer 107 may facilitate application of the medial force MF to the drape 150.

The drape 150 may be configured to at least partially transfer the medial force MF to the abdominal wall 61. Specifically, as the drape 150 may be attached to the top surface 63 of the abdominal wall 61, the drape 150 may at least partially transfer the medial force MF to the abdominal wall 61. Therefore, in some embodiments, the traction film 120 and the drape 150 may together at least partially transfer the medial force MF to the abdominal wall 61.

The dressing 200 may apply the medial force MF to the abdominal wall 61. The medial force MF applied to the abdominal wall 61 by the dressing 200 may expedite closure the primary fascia and the open abdominal wound 60. As a result, the dressing 200 may facilitate and expedite healing of the open abdominal wound 60.

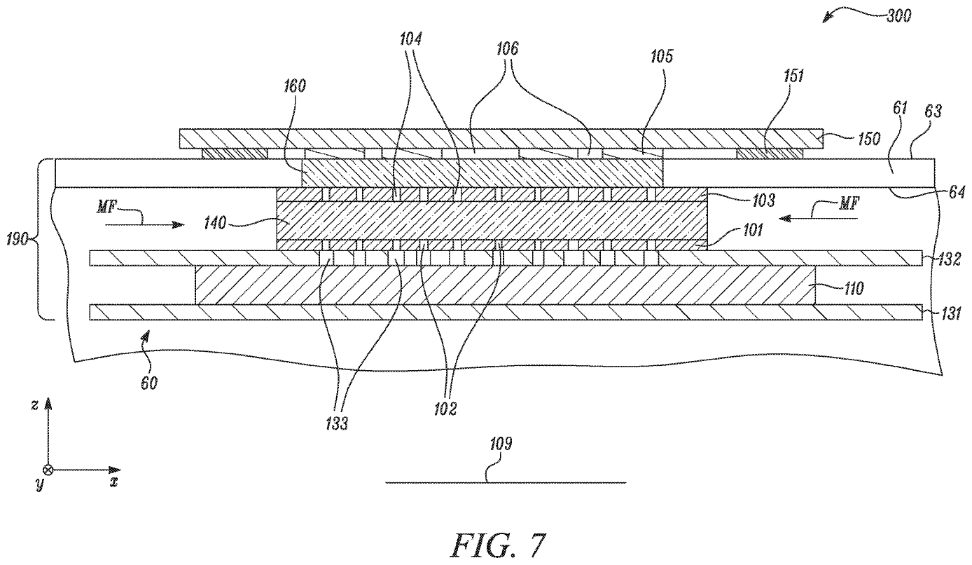

FIG. 7 illustrates a dressing 300 for the open abdominal wound 60 according to another embodiment of the present disclosure. The dressing 300 is similar to the dressing 200 of FIG. 4, with like elements designated by like reference characters. However, traction film 120 is omitted from the dressing 300. Further, the abdominal wall 61 is shown in FIG. 7 for illustrative purposes only. It may be noted that the abdominal wall 61 is not a part of the dressing 300. The tissue interface 190 of the dressing 300 does not include the abdominal wall 61.

In the illustrated embodiment of FIG. 7, the dressing 300 includes the first manifold 110, the second manifold 140, and the third manifold 160, and the drape 150. The second manifold 140 may be disposed on the first manifold 110. The third manifold 160 disposed on the second manifold 140 opposite to the first manifold 110. The drape 150 covers the third manifold 160.

The first manifold 110 may or may not include the plurality of first through openings 111 (shown in FIG. 4). The second manifold 140 may or may not include the plurality of second through openings 141 (shown in FIG. 4). The third manifold 160 may or may not include the plurality of third through openings 161 (shown in FIG. 4).

The dressing 300 further includes the first adhesive layer 101. The first adhesive layer 101 is disposed on the first manifold 110. The first adhesive layer 101 includes the plurality of first adhesive through openings 102. The first adhesive layer 101 bonds the second manifold 140 to the first manifold 110.

The dressing 300 further includes the second adhesive layer 103 disposed on the second manifold 140 opposite to the first manifold 110. The second adhesive layer 103 includes the plurality of second adhesive through openings 104. The second adhesive layer 103 bonds the third manifold 160 to the second manifold 140.

The dressing 300 further includes the third adhesive layer 105 disposed on the third manifold 160 opposite to the second manifold 140. The third adhesive layer 105 includes the plurality of third adhesive through openings 106. The third adhesive layer 105 bonds the drape 150 to the third manifold 160.

The drape 150 may be configured to be attached at least partially to the top surface 63 of the abdominal wall 61 (e.g., via the skin adhesive 151). The drape 150 is further configured to be disposed in fluid communication with the negative pressure source 25 (shown in FIG. 1) for application of the negative pressure to the third manifold 160.

In the illustrated embodiment of FIG. 7, the tissue interface 190 of the dressing 300 includes the first manifold 110, the second manifold 140, the third manifold 160, the first VPL film 131, and the second VPL film 132.

Upon application of the negative pressure, each of the first manifold 110, the second manifold 140, and the third manifold 160 may collapse in the collapse plane 109 that is substantially perpendicular to the thickness of the dressing 300, such that the medial force MF is applied to the drape 150 along the collapse plane 109. The first adhesive layer 101, the second adhesive layer 103, and the third adhesive layer 105 may facilitate application of the medial force MF to the drape 150. The drape 150 may be configured to at least partially transfer the medial force MF to the abdominal wall 61.

The dressing 300 may apply the medial force MF to the abdominal wall 61. The medial force MF applied to the abdominal wall 61 by the dressing 300 may expedite closure of the primary fascia and the open abdominal wound 60. As a result, the dressing 300 may facilitate and expedite healing of the open abdominal wound 60.

The therapy system 10 (shown in FIG. 1) may include any one of the dressings 100, 200, 300. The therapy system 10 may expedite closure of the primary fascia and the open abdominal wound 60. The therapy system 10 may facilitate and expedite healing of the open abdominal wound 60.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A dressing for an open abdominal wound, the dressing comprising:
a first manifold configured to be disposed within the open abdominal wound; and
a traction film attached at least partially to the first manifold and configured to be disposed within the open abdominal wound, the traction film comprising:
a plurality of microstructures extending away from the first manifold; and
a plurality of through openings;
wherein the plurality of microstructures are configured to engage a bottom surface of an abdominal wall around the open abdominal wound, and wherein the first manifold is configured to receive a negative pressure from a negative pressure source via the plurality of through openings of the traction film.

2. The dressing of claim 1, further comprising a drape covering the traction film and attached at least partially to the traction film, wherein the drape is configured to be attached at least partially to a top surface of the abdominal wall, and wherein the drape is further configured to be disposed in fluid communication with the negative pressure source for application of the negative pressure to the first manifold.

3. The dressing of claim 1, wherein the first manifold comprises a plurality of first through openings.

4. The dressing of claim 3, wherein the first manifold further comprises:
a central portion comprising the plurality of first through openings; and
a plurality of conduits spaced apart from each other and extending outwardly from the central portion.

5. The dressing of claim 1, further comprising:
a first visceral protective layer (VPL) film disposed on the first manifold opposite to the traction film; and
a second VPL film disposed on the first manifold opposite to the first VPL film, such that the first VPL film and the second VPL film together encapsulate the first manifold, and wherein the second VPL film comprises a plurality of film through openings.

6. (canceled)

7. The dressing of claim 1, further comprising:
a second manifold disposed between the first manifold and the traction film and configured to be disposed within the open abdominal wound; and
a third manifold disposed on the traction film opposite to the second manifold and configured to at partially engage an edge of the abdominal wall.

8. The dressing of claim 7, wherein the second manifold comprises a plurality of second through openings, and wherein the third manifold comprises a plurality of third through openings.

9. The dressing of claim 7, wherein each of the first manifold, the second manifold, and the third manifold comprises a foam.

10. The dressing of claim 7, wherein, upon application of the negative pressure, each of the first manifold, the second manifold, and the third manifold collapses in a collapse plane that is substantially perpendicular to a thickness of the dressing, such that a medial force is applied to the traction film along the collapse plane, and wherein the plurality of microstructures is configured to at least partially transfer the medial force to the abdominal wall.

11. The dressing of claim 7, wherein the plurality of microstructures at least partially surrounds the third manifold.

12. The dressing of claim 7, further comprising:
a first adhesive layer disposed between the first manifold and the second manifold, the first adhesive layer comprising a plurality of first adhesive through openings, wherein the first adhesive layer at least partially attaches the second manifold to the first manifold; and
a second adhesive layer disposed between the traction film and the third manifold, the second adhesive layer comprising a plurality of second adhesive through openings, wherein the second adhesive layer at least partially attaches the third manifold to the traction film.

13. The dressing of claim 1, wherein the plurality of microstructures comprises a plurality of sets of microstructures radially spaced apart from each other.

14. The dressing of claim 1, wherein the traction film comprises a backing layer comprising a first major surface and a second major surface opposite to the first major surface, wherein the second major surface faces the first manifold, and wherein each of the plurality of microstructures extends from the first major surface.

15. The dressing of claim 14, wherein the plurality of microstructures is integral with the backing layer.

16. (canceled)

17. The dressing of claim 1, wherein each of the plurality of microstructures comprises a height ranging from 1 micron to 1000 microns.

18. A dressing for an open abdominal wound, the dressing comprising:
a first manifold configured to be disposed within the open abdominal wound;
a first adhesive layer disposed on the first manifold and comprising a plurality of first adhesive through openings;
a second manifold configured to be disposed within the open abdominal wound, wherein the first adhesive layer bonds the second manifold to the first manifold;
a second adhesive layer disposed on the second manifold opposite to the first manifold, the second adhesive layer comprising a plurality of second adhesive through openings;
a third manifold disposed on the second manifold opposite to the first manifold and configured to at least partially engage an edge of an abdominal wall around the open abdominal wound, wherein the second adhesive layer bonds the third manifold to the second manifold;
a third adhesive layer disposed on the third manifold opposite to the second manifold, the third adhesive layer comprising a plurality of third adhesive through openings; and
a drape covering the third manifold, wherein the third adhesive layer bonds the drape to the third manifold, wherein the drape is configured to be attached at least partially to a top surface of the abdominal wall, and wherein the drape is further configured to be disposed in fluid communication with a negative pressure source for application of a negative pressure to the third manifold.

19. (canceled)

20. A therapy system for an open abdominal wound, the therapy system comprising:
a negative pressure source configured to generate a negative pressure; and
a dressing disposed in fluid communication with the negative pressure source, the dressing comprising:
a first manifold configured to be disposed within the open abdominal wound; and
a traction film attached at least partially to the first manifold and configured to be disposed within the open abdominal wound, the traction film comprising:
a plurality of microstructures extending away from the first manifold; and
a plurality of through openings;
wherein the plurality of microstructures are configured to engage a bottom surface of an abdominal wall around the open abdominal wound, and wherein the first manifold receives the negative pressure from the negative pressure source via the plurality of through openings of the traction film.

21. The therapy system of claim 20, wherein the dressing further comprises a drape covering the traction film and attached at least partially to the traction film, wherein the drape is configured to be attached at least partially to a top surface of the abdominal wall, and wherein the drape is further configured to be disposed in fluid communication with the negative pressure source for application of the negative pressure to the first manifold.

22. (canceled)

23. The therapy system of claim 20, wherein the first manifold further comprises:
a plurality of first through openings;
a central portion comprising the plurality of first through openings; and
a plurality of conduits spaced apart from each other and extending outwardly from the central portion.

24. The therapy system of claim 20, further comprising:
a first visceral protective layer (VPL) film disposed on the first manifold opposite to the traction film; and
a second VPL film disposed on the first manifold opposite to the first VPL film, such that the first VPL film and the second VPL film together encapsulate the first manifold, and wherein the second VPL film comprises a plurality of film through openings.

25. (canceled)

26. The therapy system of claim 20, wherein the dressing further comprises:
a second manifold disposed between the first manifold and the traction film and configured to be disposed within the open abdominal wound; and
a third manifold disposed on the traction film opposite to the second manifold and configured to at partially engage an edge of the abdominal wall.

27.-29. (canceled)

30. The therapy system of claim 26, wherein the plurality of microstructures at least partially surrounds the third manifold.

31. The therapy system of claim 26, further comprising:
a first adhesive layer disposed between the first manifold and the second manifold, the first adhesive layer comprising a plurality of first adhesive through openings, wherein the first adhesive layer at least partially attaches the second manifold to the first manifold; and
a second adhesive layer disposed between the traction film and the third manifold, the second adhesive layer comprising a plurality of second adhesive through openings, wherein the second adhesive layer at least partially attaches the third manifold to the traction film.

32.-36. (canceled)
